(19) United States
(12) Patent Application Publication
(10) Pub. No.: US 2005/0075312 A1
(43) Pub. Date: Apr. 7, 2005

(54) Antitumor inhibitors and use thereof

(76) Inventors: Robert J. Linhardt, Albany, NY (US); Yeong Shik Kim, Seoul (KR)

Correspondence Address: Schwegman, Lundberg, Woessner & Kluth, P.A., P.O. Box 2938, Minneapolis, MN, 55402, US

(21) Appl. No.: 10/786,613

(22) Filed: Feb. 23, 2004

Related U.S. Application Data
(60) Provisional application No. 60/449,661, filed on Feb. 24, 2003.

(51) Int. Cl. A61K 31/737

(52) U.S. Cl. 514/54

(57) ABSTRACT

The present invention provides pharmaceutical compositions for the treatment of cancer and inhibiting an increase in the volume or mass of a tumor, and methods for the treatment of cancer and inhibiting an increase in the volume or mass of a tumor.

### PRIORITY OF INVENTION

This application claims priority from U.S. Provisional Application No. 60/449,661, filed Feb. 24, 2003, which application is incorporated herein by reference.

### GOVERNMENT FUNDING

The invention described herein was made with government support under Grant Number HL52622 awarded by the National Institutes of Health. The United States Government has certain rights in the invention.

### BACKGROUND OF THE INVENTION

Angiogenesis, or neovascularization, is the formation of new capillaries from preexisting blood vessels and is a fundamental process involved in a number of physiological (Folkman, 1971; Folkman, 1972; Folkman and Shing, 1992) and pathophysiological processes (Folkman, 1995; Carmeliet and Jain, 2000). In cancer, this process contributes to the progressive growth and metastasis of solid tumors. (Liotta et al., 1991).

Tumor angiogenesis is regulated by the production of angiogenic stimulators including members of fibroblast growth factor (FGF) and vascular endothelial growth factor (VEGF) families (Colville-Nash and Willoughby, 1997; Kim et al., 1993). Drugs that interfere with angiogenesis, by halting the action of angiogenic proteins, might reduce the size of tumors and maintain them in a dormant state. Angiogenic inhibitors such as angiostatin and endostatin can modulate angiogenesis both at the primary site and at the downstream sites of metastasis (O'Reilly et al, 1994, 1997). The potential use of these and other natural and synthetic angiogenesis inhibitors is currently being studied intensively by many laboratories (Mohan et al., 2000; Suh et al., 1997; Minamiguchi et al., 2001; Kim et al., 2000). Such agents may have reduced toxicity and may be less likely to generate drug resistance than conventional cytotoxic drugs (Keshet et al., 1999).

Heparin/heparan sulfate interacts with various angiogenic growth factors (Capila and Linhardt, 2002). Angiogenic growth factors induce response in target endothelial cells by binding to cognate cell-surface tyrosine kinase receptors (Gale and Yancopoulos, 1999). The interaction of heparin-binding growth factors to tyrosine kinase receptors is modulated by heparan sulfate proteoglycans. Acharan sulfate (AS) isolated from the giant African snail, Achatina fulica, is a novel member of glycosaminoglycan (GAG) family (Kim et al., 1996). This GAG has a major repeating disaccharide structure of →4)-α-D-GlcNpAc(1→4)-α-L-IdoAp2S(1→, where GlcNpAc is 2-acetamido 2-deoxyglucopyranose, IdoAp is idopyranosyluronic acid and S is sulfate (FIG. 1). This polysaccharide has a molecular weight of 135,000, when calculated by HPLC-GPC analysis. Recently, we observed that AS interfered with heparin's bFGF mitogenicity in vitro, suggesting its possible utility as an angiogenesis inhibitor (Wang et al., 1997).

U.S. Pat. No. 6,028,061 describes and claims the use of AS in inhibiting angiogenesis based on its inhibition of FGF (fibroblast growth hormone). We have now discovered that AS has antitumor activity as demonstrated in both in vivo and in vitro assays. The in vivo antitumor activity is demonstrated against the sarcoma 180-induced solid tumor and primary tumor in LLC-bearing C57BL/6 mice. This is the first demonstration of in vivo antitumor activity using AS ever observed. Although more than 30 years ago it was hypothesized that tumor growth is angiogenesis dependent (Folkman, 1971) anti-angiogenesis activity does not predict in vivo tumor growth inhibition. Thus, the present invention provides a marked advance in the elucidation of useful in vivo anti-tumor agents.

### SUMMARY OF THE INVENTION

The present invention provides pharmaceutical compositions for the treatment of cancer and for inhibiting an increase in the volume or mass of a tumor in a host in need of treatment. The present invention also provides methods for the treatment of cancer and for the inhibition of an increase in the volume or mass of a tumor in a host in need of treatment. Compounds which are the active ingredients of the compositions and methods of the present invention are represented by the formula
[→4)-α-D-GlcNpAc(1→4)-α-L-IdoAp2S(1→]n Formula I
wherein GlcNpAc is 2-acetamido 2-deoxyglucopyranose, IdoAp is idopyranosyluronic acid and S is sulfate, and n is 1 to 1000.

The present invention is based on the discovery that acharan sulfate demonstrates in vivo anti-tumor activity. Thus, a novel method of inhibiting tumor growth and treating cancer is provided by the present invention. As used herein acharan sulfate means a glycosaminoglycan from the giant African snail, Achatina fulica having primarily the repeating disaccharide structure of α-D-N-acetylglucosaminyl 2-sulfoiduronic acid as depicted by the above Formula I. See also FIG. 1.

### BRIEF DESCRIPTION OF THE FIGURES

FIG. 1 depicts the structure of acharan sulfate. And the site of its structure where it can be cleaved using heparin lyase II.

FIG. 2 is a photograph of a control egg and one treated with acharan sulfate showing the effect of acharan sulfate on inhibition of angiogenesis.

FIG. 3 shows the effect of acharan sulfate on bFGF-induced angiogenesis in mouse model.

FIG. 4 shows the effect of acharan sulfate on calf pulmonary endothelial cell proliferation by MTT assay.

FIG. 5 shows the effect of acharan sulfate on tumor volume in Lewis lung carcinoma-bearing mice.

FIG. 6 shows the effect of acharan sulfate on tumor weight in Lewis lung carcinoma-bearing mice.

FIG. 7 shows effects of acharan sulfate on tumor volume (A) and tumor weight (B) in sarcoma 180-bearing mice.

FIG. 8 shows effect of acharan sulfate on survival time of mice sarcoma with 180 ascitic tumor.

### DETAILED DESCRIPTION

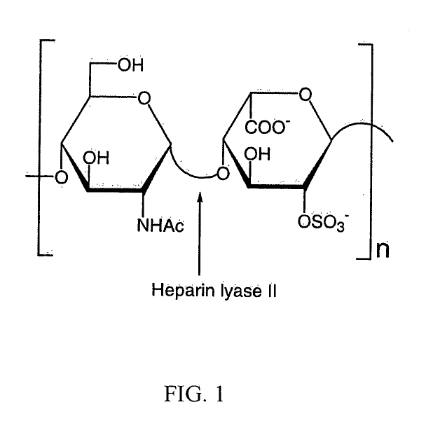

Acharan sulfate is a glucosaminoglycan having a repeating disaccharide structure described as →4)-α-D-GlcNpAc(1→4)-α-L-IdoAp2S(1→, where GlcNpAc is 2-acetamido 2-deoxyglucopyranose, IdoAp is idopyranosyluronic acid and S is sulfate (FIG. 1).

The present invention is directed to the use of and pharmaceutical compositions comprising compounds of the formula
[→4)-α-D-GlcNpAc(1→4)-α-L-IdoAp2S(1→]n Formula I
wherein GlcNpAc is 2-acetamido 2-deoxyglucopyranose, IdoAp is idopyranosyluronic acid and S is sulfate, and n is 1 to 1000.

In the above Formula I n is preferably 4 to 500 and more preferably 51 to 100. Another preferred embodiment of the present invention is the use of and compositions comprising compounds of Formula I for the treatment of cancer or for inhibiting an increase in the mass or volume of a tumor in a patient in need of treatment wherein n is 4 to 100 and more preferably 4 to 50. The pharmaceutical compositions of the present invention are useful in the treatment of cancer and in the inhibition of an increase in the volume or mass of a tumor in a patient in need of treatment. The use of the compounds of Formula I is directed to a method of treating cancer and of inhibiting an increase in the mass or volume of a tumor in a patient in need of treatment.

Described herein are experiments carried out to evaluate the antiangiogenic activity of acharan sulfate. We also show herein that acharan sulfate inhibits new blood vessel formation in the in vivo matrigel and chorioallantoic membrane assays. Additionally, we show that acharan sulfate has substantial antitumor activity against sarcoma 180-induced solid and primary tumors in Lewis lung carcinoma-bearing C57BL/6 mice.

### EXAMPLE 1

### Preparation of Acharan Sulfate

Acharan sulfate was isolated from the soft body tissue of the giant African snail by proteolysis of defatted tissue and purified by fractional precipitation and ion-exchange chromatography as previously described (Kim et al., 1996; Jeong et al., 2001). In brief, five hundred milligrams of the crude sample was dissolved in 50 mL of 50 mM sodium phosphate buffer (pH 7.0) and applied to a column (2.5×50 cm) of DEAE-Sepharose equilibriated in the same buffer. The column was eluted in a stepwise gradient with 50 mM sodium phosphate buffer containing 0.0 M, 0.5 M and 1.0 M NaCl. The elution was monitored at 210 nm and the flow rate was set at 30 mL/h. Each fraction was collected, dialyzed and freeze-dried to give a white powder. All samples were subjected to ¹H-NMR spectroscopy and agarose gel-electrophoresis. Simultaneously, the fractions were depolymerized by heparin lyase II and the reaction products were analyzed by strong anion-exchange (SAX)-HPLC as previously described (Jeong et al., 2001). The average of molecular weight was determined by gel-permeation chromatography (GPC)—HPLC using a TSK 6000PW column (Torrance, Calif., U.S.A.).

### Characterization of Acharan Sulfate

Carbazole assay of the polysaccharide eluted at 1.0 M NaCl from DEAE-Sepharose ion-exchange chromatography showed that it contained uronic acid (Jeong et al., 2001). Azure A dye binding assay demonstrated the presence of sulfate groups in the structure being consistent with a glycosaminoglycan. ¹H-NMR analysis of the intact polysaccharide demonstrated the presence of two anomeric protons having chemical shifts corresponding to the H-1 of GlcNpAc at δ5.1 and H-1 of IdoAp2S at δ5.2, respectively. The observation of this upfield shift of the anomeric proton of GlcNpAc is attributable to the unusual 4)-α-D-GlcNpAc(1→4)-α-L-IdoAp2S(1→sequence of AS (Kim et al., 1996). The fact that this fraction was not sensitive to chondroitinase ABC strongly indicated that it is entirely composed of a new glycosaminoglycan. The depolymerized product by heparinase II also contained a repeating disaccharide unit of ΔUAp2S-GlcNpAcα,β more than 95%, where ΔUAp is 4-deoxy-α-L-threo-hex-4-enopyranosyluronic acid. The average molecular weight of a new glycosaminoglycan was determined to be 135,000 by GPC-HPLC. The average number of disaccharide unit (n) is approximately 300. Heparin lyase II can cleave the α1→4 linkage giving a repeating disaccharide unit.

The invention provides compositions and methods that can be used to treat cancer utilizing the compounds of Formula I. These compounds are shown herein to inhibit a gain in mass or volume of a tumor. Accordingly, these compounds may be administered to an animal in need of such treatment, including warm blooded animals, such as a human. The compounds can be administered alone, as pharmaceutical compositions, or in conjunction with other therapeutic agents that are known in the art. Furthermore, these compounds may be formulated as pharmaceutical dosage forms containing an effective amount of the compound to inhibit tumors from gaining mass or volume. In addition, the compounds of the invention can be formulated as single unit dosage forms. A typical tablet formulation would include a compound of Formula I, compounded with lactose, povidone, croscarmellose sodium, microcrystalline cellusose and magnesium stearate.

These compounds are acidic and thus would be typically used as salts. Examples of acceptable salts are formed through the addition of metallic or ammonium bases to form physiological acceptable salts, for example, sodium, potassium, calcium, ammonium, alkylammonium and arylammonium salts.

Acceptable salts may be obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The effective dosage of a compound of Formulae I for inhibition of an increase in the volume or mass of a tumor or as an anticancer agent is extrapolated from the results of the in vivo studies set forth herein. The effective dosage is dependent not only on the particular compound employed, but also, on the method of administering the compound. The means of delivery may be topical, including buccal and sublingual, oral, subcutaneous, intranasal, intravaginal, rectal, intramuscular, intraperitoneal, intradermal, or intravenous. Administration of the active ingredient may also be achieved by using a biodegradable, polymeric implant. The formulations may conveniently be presented in unit dosage forms, e.g., tablets and sustained release capsules, and in liposomes, and may be prepared by any methods well known in the art of pharmacy. See, for example, Remington's Pharmaceutical Sciences. U.S. Pat. No. 6,028,061 describes methods useful in formulating the compounds and compositions of the present invention as set forth hereinbelow.

Such preparative methods include the step of bringing into association with the molecule to be administered ingredients such as the carrier which constitutes one or more accessory ingredients. In general, the compositions are prepared by uniformly and intimately bringing into association the active ingredients with liquid carriers, liposomes or finely divided solid carriers or both, and then if necessary shaping the product.

Compositions of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion, or packed in liposomes and as a bolus, etc.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface-active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein.

Compositions suitable for topical administration include lozenges comprising the ingredients in a flavored basis usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers and may be stored in a freeze dried condition requiring only the addition of the sterile liquid carrier prior to use.

In demonstrating the utility of the compounds and compositions of the present invention the following materials were used. Trypan blue solution (0.4%), HEPES, methylthiazol-2-yl-2,5-diphenyl-tetrazolum bromide (MTT), phosphate-buffered saline (PBS), DEAE-Sepharose fast flow, heparin, Drabkin's reagent kit 525, p-nitrophenyl phosphate, 5-fluorouracil (5-FU), and all-trans-retinoic acid were purchased from Sigma (St. Louis, Mo., USA). Trypsin-EDTA, penicillin-streptomycin, fetal bovine serum (FBS), Dulbecco's modified Eagle's medium (DMEM), HBSS, Roosevelt Park Memorial Institute Medium (RPMI) 1640 medium, and basic fibroblast growth factor (bFGF) were from GIBCO/BRL Gaithersburg, Md., USA). Alcalase was from Novo Korea (Seoul, Korea). Doxorubicin was kindly provided from Boryung Pharmaceuticals (Seoul, Korea).

The animals used herein are as follows.

Seven-week-old, specific pathogen-free (SPF) male C57BL/6J mice were supplied from Japan SLC, Inc. (Shizuoka, Japan) for the matrigel plug assay. Five-week-old, SPF male C57BL/6Ntac were purchased from Samtaco BioKorea (Osan, Korea). SPF ICR mice were purchased from Daehan Biolink Co. (Umsung, Korea). All animal work was carried out in a pathogen-free barrier zone at Seoul National University Hospital in accordance with the procedure outlined in the Guide for the Care and Use of Laboratory Animals. Animals were fed sterilized animal chow and water ad libitum and they were housed at 23±0.5° C., 10% humidity in a 12-h light-dark cycle.

The cell cultures used herein are the following.

Lewis lung carcinoma cells (American Type Cell Collection, Rockville, Md.) were maintained in DMEM supplemented with heat-inactivated 10% FBS (Life Technologies, Grand Island, N.Y.), 100 units/ml penicillin, and 100 μg/ml streptomycin. Calf pulmonary arterial endothelial (CPAE) cells and sarcoma 180 (Korea Cell Line Bank, Seoul, Korea) were cultured in RPMI 1640 media containing 10% FBS and 1% antibiotics in a 37° C. incubator with a humidified atmosphere containing 5% CO₂. The viable cells alone were counted with a hemocytometer using the trypan blue dye exclusion test (Kaltenbach et al., 1958).

The effect of acharan sulfate on the inhibition of angiogenesis was performed using the choroioallantoic membrane (CAM) assay. These assays essentially followed previously published procedures (Tanaka et al, 1986; Oikawa et al., 1990). In brief, fertilized chicken eggs were incubated in the constant humidified breeder at 37° C. On the third day of incubation, about 2 ml of egg albumin were aspirated by an 18-gauge hypodermic needle, to detach the developing CAM from the shell. One and half days later, sample-loaded thermanox coverslips (Nunc, Naperville, Ill.) were air-dried and applied to the CAM surface for testing of angiogenesis inhibition by AS. Two days later, 1 ml of 10% fat emulsion (Intralipose) was injected into the chorioallantoic membrane and the avascular zone was observed under a dissecting microscope. Inhibition of angiogenesis was assessed when the avascular zone exceeded 3 mm. In order to abolish the possibility of contaminant in AS, the depolymerized product by heparinase II as described above was also tested for the antiangiogenic activity. The concentration of AS used in this assay was selected based on the concentration of heparin that had previously been applied in the same assay (Collen et al., 2000).

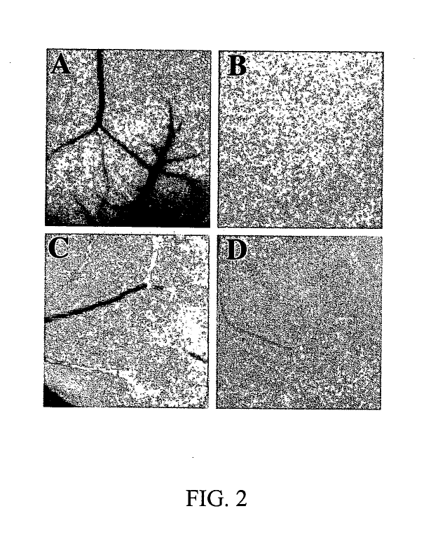

On day 4.5, CAMs were treated with different doses of AS for 2 days. The dose-response relationship for the appearance of avascular zone was determined. The inhibitory effect of AS on the treated CAM is shown in FIG. 2 and Table 1.

TABLE 1
Effect of AS on chick embryonic angiogenesis

| | Concen- | Eggs showing | | |
|---|---|---|---|---|
| | tration | antiangio- | Total eggs | |
| Compounds | (μg/egg) | genesis^{a} | tested | % inhibition |
| Control (H₂O) | — | 3 | 54 | 5.6 |
| Retinoic acid^{b} | 1 | 20 | 25 | 80.0 |
| Acharan sulfate | 50 | 2 | 40 | 5.0 |
| depolymerization | | | | |
| mixture | | | | |
| Acharan sulfate I | 5 | 14 | 29 | 48.3 |
| Acharan sulfate II | 10 | 15 | 27 | 55.6 |

^{a}Antiangiogeneis was assessed when the avascular zone exceeded 3 mm.
^{b}Retinoic acid was used as a positive control.

In FIG. 2, A) is negative control (water); B) is retinoic acid (1 μg/ml); C) is depolymerization mixture of AS by heparinase 11 (50 μg/ml); D) is intact AS (10 μg/ml)) (Magnification 2×).

Compared to the effect of vehicle as control, which did not have antiangiogenic activity in the treated CAM, AS at doses of 5 and 10 μg/pellet showed antiangiogenic activity of 48.3 and 55.6%, respectively. The effect of AS on chick embryonic angiogenesis decreased in a dose-dependent fashion. Retinoic acid strongly inhibited angiogenesis (80%) even at 1 μg/egg, but it may have a toxic effect to cells. The depolymerization mixture of AS by heparin lyase II did not cause any inhibition of angiogenesis, indicating that any contaminant in the intact AS could not act as an angiogenic inhibitor.

The effect of acharan sulfate on the inhibition of angiogenesis was also evaluated in the matrigel plug assay. This assay was performed as previously described (Passaniti et al., 1992). Acharan sulfate, dissolved in water, bFGF and heparin, dissolved in 0.1% bovine serum albumen (BSA)/phosphate buffered saline (PBS) were mixed with liquid matrigel (Collaborative Biomedical Products, Bedford, Mass.) in proportions not exceeding 1% of the total volume of matrigel. A mixture of 0.5 ml matrigel with AS or vehicle was injected subcutaneously into C57BL/6J mice. After injection, the matrigel rapidly formed a plug. Seven days later, the skin of the mouse was easily pulled to expose the matrigel plug, which remained intact. The amount of hemoglobin (Hb) inside the matrigel was measured using the Drabkin method (Drabkin and Austin, 1932) and Drabkin reagent 525 for the quantitation of blood vessel formation. The concentration of Hb was calculated based on a Hb standard measured simultaneously.

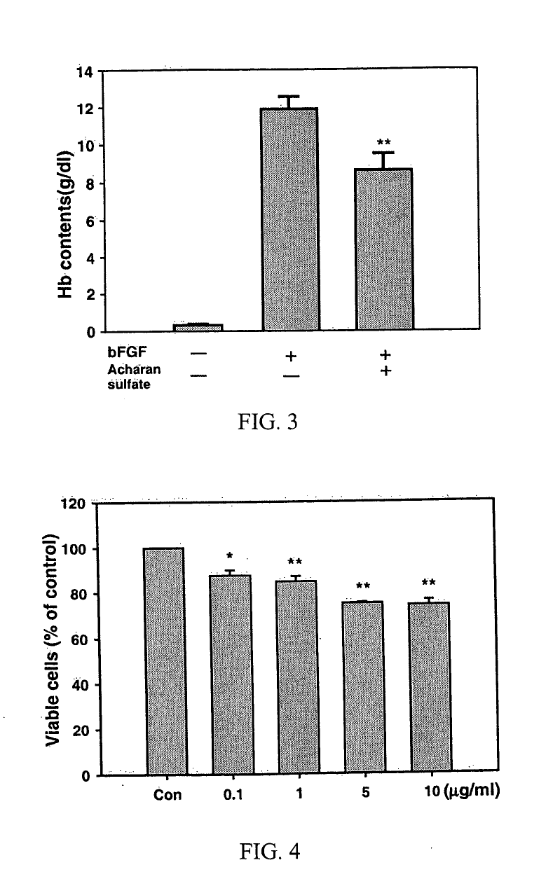

To evaluate the effect of AS on ongoing angiogenic process in the mouse matrigel plug assay matrigel, heparin (10 units/500 μl), and bFGF (100 ng/500 μl) with or without AS were injected s.c. into C57BL/6 mice. Seven days later, matrigel plug was excised to allow clear visualization of the intact vessels of the matrigel. The control samples in the matrigel assay had no vessels. A combination with 100 ng/ml bFGF and 10 units/ml (˜65 μg/ml) of heparin pulled many vessels from the surrounding tissues into the matrigel. The new vessels were abundantly filled with intact red blood cells, indicating the formation of a functional vasculature inside the matrigel and blood circulation in newly formed vessels by angiogenesis induced by bFGF and heparin. Fifty micrograms of AS in combination with bFGF and heparin slightly prevented the vessel induction, indicating that AS suppressed the bFGF-stimulated angiogenesis. We next measured the hemoglobin content inside the matrigel plugs to quantify the angiogenesis. Whereas bFGF and heparin increased Hb concentration to 11.8 g/dl and the Hb concentration inside the control was 0.3 g/dl, AS decreased the heparin and bFGF-elevated Hb quantity to about 8.6 g/dl (FIG. 3). Each value represents mean±S.E.M. of at least 5 animals. The data are significantly different from the control; **P<0.01 Anti-angiogenesis in this assay did not result from the effect of a vehicle of bFGF and the injection sites showed no signs of inflammation and hemorrhage. Anti-angiogenesis in this assay did not result from the effect of a vehicle of bFGF and the injection sites showed no signs of inflammation and hemorrhage.

The effect of acharan sulfate on in vitro cell proliferation was carried out using calf pulmonary artery endothelial (CPAE) cells as follows.

CPAE cells were seeded in a 24 well plate at a cell density of 10⁵ cells/well in 90% RPMI and 10% FBS. After 24 h incubation, the cells were treated with various concentrations of AS. Three days later, new media and MTT solution were added to each well. After incubation at 37° C. for 4 h, the absorbance of treated cells at 540 nm was compared to that of control cells.

Using increasing concentrations of AS on CPAE cell viability, AS showed no cytotoxic effect on CPAE cells (data not shown). We, then, examined the effect of AS on the proliferation of CPAE cells by MTT assay. AS exhibited an inhibitory effect in a concentration-dependent fashion. After a 3-day treatment, growth inhibition of 12.5%, 15.2%, and 24.9% was observed at AS concentrations as low as 0.1, 1 and 10 μg/ml, respectively (FIG. 4). The data are significantly different from the control; *p<0.05, **P<0.01.

The effect of AS in vivo on tumor growth was evaluated as follows.

Male C57BL/6 mice were inoculated s.c. in the back with LLC cells (1×10⁶/animal) on day 0. After tumor volume was at least 60-100 mm³, AS was administered into the subcutaneous region near the tumor mass at two doses of 10 and 30 mg/kg for 15 days. The size of tumors in all groups was measured using a dial-caliper and the volume of tumors was determined using the formula width²×length×0.52 (Voest et al., 1995; Cao et al., 1995). The effects of AS on tumor growth and host survival were also measured by evaluating tumor volumes, tumor weights and percentage increase in lifespan of tumor hosts, respectively (Oguchi et al., 1987; Kusumoto, 1991). For calculating the survival time, mice were inoculated i.p. with 106 sarcoma 180 cells/mouse on day 0 and the treatment with two doses of AS (50 and 100 mg/kg, i.p.) were started 24 h after inoculation for nine consecutive days. The control group was treated with saline. Median survival time (MST) for each group (n=7) was observed and the antitumor activity of the test compounds were compared with that of control group by measuring the increase in lifespan.

For solid tumor development, ICR mice were injected with 0.1 ml of sarcoma 180 suspensions into the right hind limbs. After 6 days of tumor transplantation, mice randomized into six groups were injected i.p. with AS (50 and 100 mg/kg) and 5-FU (25 mg/kg) once a day for 9 days. Eight days later after treatment, animals were sacrificed by cervical dissociation, and solid tumors were removed and weighed.

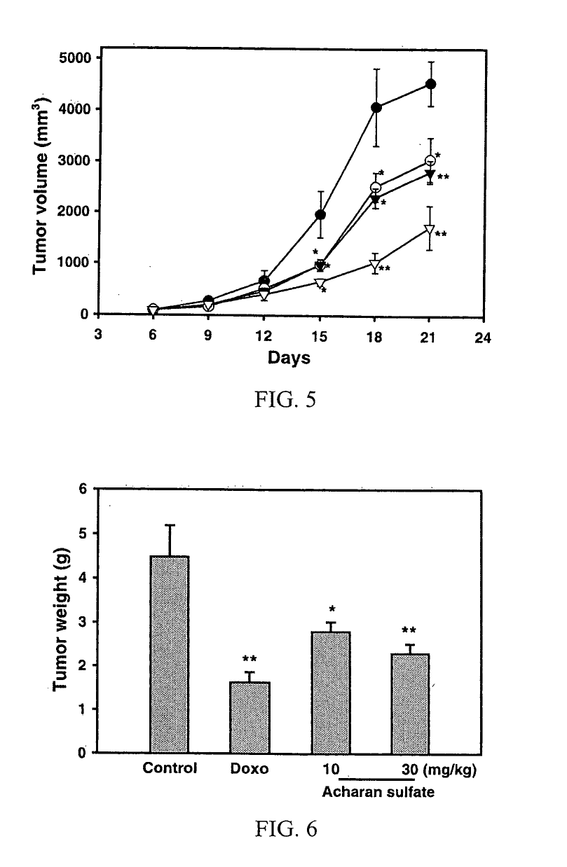

The results of AS on tumor growth in C57BL/6 mice inoculated with Lewis lung carcinoma cells are shown in FIGS. 5 and 6. A daily subcutaneous injection of 10 and 30 mg/kg suppressed the growth of primary tumors during the 15-day treatment course. At the end of treatment, tumor growth was inhibited by 32.8% (3049.2 mm³) and 38.1% (2809.3 mm³), respectively at a dose of 10 mg/kg and 30 mg/kg, as compared to control mice treated with saline alone (4534.4 mm³). In contrast, tumor grew rapidly to sizes >4000 mm³ in saline-treated mice during the same 15-day treatment period. Doxorubicin as positive control was administered i.v. every five day at a dose of 10 mg/kg. It inhibited tumor growth by 62.0% (1721.6 mm³). The AS-treated mice did not lose weight over the course of treatment, indicating that AS showed little or no toxicity. On day 21, tumor tissues were removed and weighed. It was found that the tumor weight was reduced dose-dependently by the injection of AS as shown in FIG. 6. A mean tumor weight reductions by 37.8% (2.8±0.2 g) at 10 mg/kg and by 48.9% (2.3±0.2 g) at 30 mg/kg were observed, compared with the saline group (4.5±0.7 g). Doxorubicin significantly reduced the tumor weight by 68.0% (1.6±0.2 g). However, the loss of weight in the group of the doxorubicin-treated mice was marked as compared with that of the control mice and the injection area was significantly damaged over the treatment. In FIG. 5 the data are significantly different from control group; *p<0.05, **p<0.01. ● saline; ∘ AS 10 mg/kg; ▾ AS 30 mg/kg; □ Doxorubicin 10 mg/kg. In FIG. 6 the data is significantly different from the control; *p<0.05, **p<0.01.

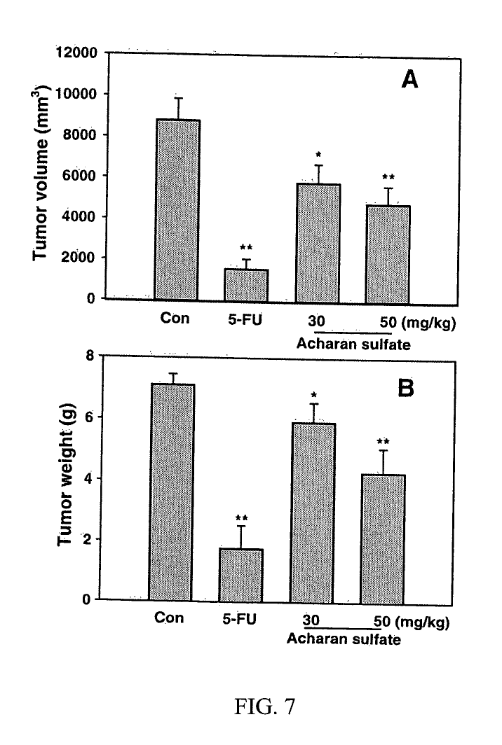

The results of the effect of AS on solid tumor induced by sarcoma 180 tumor cells in ICR are shown in FIG. 7. As shown in FIG. 7A, the average tumor volume in the control was 8804±465.3 mm³. The level of the tumor volume in groups treated with 5-FU injection decreased by 82.1% (1572±201.5 mm³), compared with the control level. AS at the dose of 50 mg/kg inhibited the tumor volume by 45.0% (4799±345.2 mm³). AS at the dose of 50 mg/kg inhibited the tumor weight by 39.6% (4.3±0.1 g), while 5-FU at the dose of 25 mg/kg inhibited the tumor weight by 75.1% (1.8±0.3 g) and 55.8% (3.1±0.3 g), compared with the control (7.1±0.1 g) (FIG. 7B). The data were presented as mean±S.E.M. of nine mice. Significantly different from the control; *p<0.05, **p<0.01.

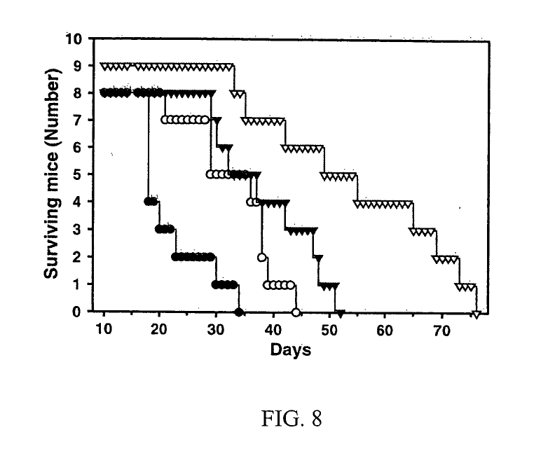

The results of the effect of AS on the survival time in sarcoma 180 bearing mice are summarized in FIG. 8. The median survival time in the control was 22.4±2.2 days, while it was dose-dependently increased on the treatments of AS at two doses of 30 and 50 mg/kg/day for 9 consecutive days. AS showed that the lifespan was increased by 1.5-folds (34.3±2.6 days) against the control group at the dose of 30 mg/kg, while its lifespan was prolonged by 1.8-fold on treatment of 50 mg/kg (40.6±3.1 days) (FIG. 8). The animal group treated with 25 mg/kg dose of 5-FU, as a positive control, showed a much stronger enhancement of MST (42.8±4.2 days). The symbols shown in this figure correspond to ● control, ∘ AS 30 mg/kg, ▾ AS 50 mg/kg, □ 5-FU.

All data are presented as mean±S.E. or as percentage to control. Statistical comparisons between groups were performed using the Student's t test. The values at p<0.01 and p<0.05 were considered statistically to be significant.

The foregoing results show that acharan sulfate acts as angiogenesis inhibitor and in an antitumor agent in vivo. Based on the above results AS does not influence proliferation of endothelial cells as demonstrated in the CPAE test. Additionally using the CAM assay the above results show that AS markedly inhibits the development of capillary networks at two concentrations (5 and 10 μg/CAM). Further the antiangiogenic activity of AS was confirmed by performing in vivo mouse matrigel plug assay. AS inhibited the formation of neovessels induced by a combination of bFGF and heparin in matrigel. In the foregoing experiments to evaluate the antitumor effect of AS in mice bearing murine LLC tumors, AS was given by daily subcutaneous injections at a site distant from the primary tumor. We speculated that one of the mechanisms for the antiangiogenic action of AS might be the suppression of matrix metalloprotease activity. However, AS shows no detectable antiprotease activity. AS also shows substantial antitumor activity against sarcoma 180-induced solid tumor and primary tumor in LLC-bearing C57BL/6 mice. A remarkable increase in lifespan was observed in sarcoma 180 ascitic tumor. Ascites fluids are direct nutritional sources for tumor cells.

### REFERENCES

Blackhall F. H., Merry C. L., Davies E. J., Jayson G. C., 1991. Heparan sulfate proteoglycans and cancer. Br. J. Cancer 85, 1094-8.
Cao, Y., Chen, C., Weatherbee, J. A., Tsang, M., Folkman, J., 1995. Gro-β, a C—X—C chemokines, is an angiogenesis inhibitor that suppresses the growth of Lewis lung carcinoma in mice. J. Exp. Med. 182, 2069-2077.
Capila, I., Linhardt, R. J., 2002. Heparin-protein interactions. Angew. Chemie. 41, 390-412.
Carmeliet, P., Jain, R. K., 2000. Angiogenesis in cancer and other diseases. Nature 407, 249-257.
Collen, A., Smorenburg, S. M., Peters, E., Lupu, F., Koolwijk, P., Van Noorden C, Van Hinsbergh, V. W., 2000. Unfractionated and low molecular weight heparin affect fibrin structure and angiogenesis in vitro. Cancer Res. 60, 6196-6200.
Colville-Nash, P. R., Willoughby, D. A., 1997. Growth factors in angiogenesis: current interest and therapeutic potential. Mol. Med. Today. 3, 14-23.
Drabkin, D. L., Austin, J. H., 1932. Spectrophotometric constants for common hemoglobin derivatives in human, dog, and rabbit blood. J. Biol. Chem. 98, 719-722.
Folkman, J., 1971. Tumor Angiogenesis: therapeutic implications. N. Engl. J. Med. 285, 1182-1186.
Folkman, J., 1972. Anti-angiogenesis: new concept for therapy of solid tumors. Ann. Surg. 175, 409-416.
Folkman, J., 1995. Angiogenesis in cancer, vascular, rheumatoid and other disease. Nature Med. 1, 27-31.
Folkman, J., Shing, Y., 1992. Angiogenesis. J. Biol. Chem. 267, 10931-10934.
Gale, N. W., Yancopoulos, G. D., 1999. Growth factors acting via endothelial cell-specific receptor tyrosine kinases: VEGFs, angiopoietins, and ephrins in vascular development. Genes Dev. 13, 1055-1066.
Jeong, J. A. Toida, T., Muneta, Y., Kosiishi, I., Imanari, T., Linhardt, R. J., Choi, H. S., Wu, S. J., Kim, Y. S., 2001. Localization and characterization of acharan sulfate in the body of the giant African snail Achatinafulica. Comp. Biochem. Physiol. 130, 513-519.
Kaltenbach, J. P., Kaltenbach, M. H., Lyons, W. B., 1958. Dyes for differentiating live and dead ascites cells. Exp. Cell Res. 15, 112-117.
Keshet, E., Ben-Sasson, S. A., 1999. Anticancer drug targets: approaching angiogenesis. J. Clin. Invest. 104, 1497-1501.
Kim, K. J., Li, B., Winer J., Armanini, M., Gillett, N., Phillips, H. S., Ferrara, N., 1993. Inhibition of vascular endothelial growth factor induced angiogenesis suppresses tumor growth in vivo. Nature 362, 841-844.
Kim M. S., Lee, Y. M., Moon, E. J., Kim, S. E., Lee, J. J., Kim, K. W., 2000. Anti-angiogetic activity of torilin, a sesquiterpene compound isolated from Torilis japonica. Int. J. Cancer. 87, 269-275.
Kim, Y. S., Chang, I. M., Toida, T., Park, Y., Linhardt, R. J., 1996. A new glycosaminoglycan from the giant African snail Achatina fulica. J. Biol. Chem. 271, 11750-11755.
Kusumoto, T., Maehara, Y., Sugimachi, Y., Kohnos, S., Sugimachi, K., 1991. Hyperthermia potentiates the cytotoxic activity of 1-hexylcarbamoyl-5-fluorouracil in sarcoma-180 cells and human malignant tumor cells. Anticancer Res. 11, 1963-1966.
Liotta, L. A., Steeg, P. S., Stetler-Stevenson, W. G., 1991. Cancer metastasis and angiogenesis: an imbalance of positive and negative regulation. Cell 64, 327-336.
Minamiguchi k., Kumagai H., Masuda T., Kawada M., Ishizuka M., Takeuchitin T., 2001. Thiolutin, an inhibitor of HUVEC adhesion to vitronectin, reduces paxillin in HUVEC and suppresses tumor cell-induced angiogenesis. Int. J. Cancer. 93, 307-316.
Mohan, R., Sivak, J., Ashton, P., Russo, L. A., Pham, B. Q., Kasahara, N., Raizman, N. B., Fini, M. E., 2000. Curcuminoids inhibit the angiogenic response stimulated by fibroblast growth factor-2, including expression of matrix metalloproteinase gelatinase B. J. Biol. Chem. 275, 10405-10412.
Oguchi, T., Ando, T., Matsunaga, K., Fujii, T., Yoshikumi, C., Nomoto, K., 1987. Effect of PK on cytotoxicity against sarcoma-180 in tumor-bearing mice. Anticancer Res. 7, 481-484.
Oikawa, T., Hirotani, K., Ogasawara, H., Katayama, T., Nakamura, O., Iwaguchi, T., Hiragun, A., 1990. Inhibition of angiogenesis by vitamin D3 analogue. Eur. J. Pharmacol. 178, 247-250.
O'Reilly, M. S., Boehm, T., Shing, Y., Folkman, J., 1997. Endostatin: an endogenous inhibitor of angiogenesis and tumor growth. Cell 88, 277-285.
O'Reilly, M. S., Holmgren, L., Shing, Y., Chen, C., Folkman, J., 1994. Angiostatin: A novel angiogenesis inhibitor that mediates the suppression of metastases by a Lewis lung carcinoma. Cell 79, 315-328.
Passaniti, A., Taylor, R. W., Pili, R., Guo, Y., Long, P. V., Haney, J. A., Pauly, R. R., Grant, D. S., Martin G. R., 1992. Simple and quantitative method for assessing antiangiogenesis and antiangiogenic agents using reconstituted basement membrane, heparin, and fibroblast growth factor. Lab. Invest. 69, 519-528.
Sasisekharan, R., Shriver, Z., Venkataraman, G., Narayanasami, U., 2002, Roles of heparan sulfate glycosaminoglycans in cancer. Nat. Rev. Cancer 2, 521-528
Suh, H. S., Jung, E. J., Kim, T. H., Lee, H. Y., Park Y. H., Kim, K. W., 1997. Anti-angiogenic activity of ursodeoxycholic acid and its derivatives. Cancer Lett. 113, 117-122.
Tanaka, N. G., Sakamoto, A., Tohgo, Y., Nishiyama, T., Ogawa, H., 1986. Inhibitory effects of anti-angiogenic agents on neovascularization and growth of the choriollantoic membrane (CAM)—The possibility of a new CAM assay for angiogenesis inhibitor. Exp. Pathol. 30, 143-150.
Voest, E. E., Benyon, B. M., O'Reilly, M. S., Truitt, G., D'Amato, R. J., Folkman, J., 1995. Inhibition of angiogenesis in vivo by interleukin 12. J. Nat. Cancer Inst. 87, 581-586.

1. A method of treating cancer in a host in need of treatment comprising administering to said host an anti-cancer effective amount of a compound of the formula
[→4)-α-D-GlcNpAc(1→4)-α-L-IdoAp2S(1→]n
wherein GlcNpAc is 2-acetamido 2-deoxyglucopyranose, IdoAp is idopyranosyluronic acid and S is sulfate, and n is 1 to 1000.

2. The method of claim 1 wherein n is 4 to 500.

3. The method of claim 1 wherein n is 51 to 100.

4. The method of claim 1 wherein n is 4 to 100.

5. The method of claim 1 wherein n is 4 to 50.

6. A method of treating a host by inhibiting an increase in the volume or mass of a tumor in said host in need of treatment which comprises administering to said host a compound of the formula
[→4)-α-D-GlcNpAc(1→4)-α-L-IdoAp2S(1→]n
wherein GlcNpAc is 2-acetamido 2-deoxyglucopyranose, IdoAp is idopyranosyluronic acid and S is sulfate, and n is 1 to 1000 in an amount effective to inhibit an increase in the volume or mass of a tumor.

7. The method of claim 6 wherein n is 4 to 500.

8. The method of claim 6 wherein n is 51 to 100.

9. The method of claim 6 wherein n is 4 to 100.

10. The method of claim 6 wherein n is 4 to 50.

11. A pharmaceutical composition comprising a compound of the formula
[→4)-α-D-GlcNpAc(1→4)-α-L-IdoAp2S(1→]ₙ
wherein GlcNpAc is 2-acetamido 2-deoxyglucopyranose, IdoAp is idopyranosyluronic acid and S is sulfate, and n is 1 to 1000 in an amount effective to treat cancer in a host by inhibiting cancer growth in said host

12. The composition of claim 11 wherein n is 4 to 500.

13. The composition of claim 11 wherein n is 51 to 100.

14. The composition of claim 11 wherein n is 4 to 100.

15. The composition of claim 11 wherein n is 4 to 50.

16. A pharmaceutical composition comprising an amount of compound of the formula
[→4)-α-D-GlcNpAc(1→4)-α-L-IdoAp2S(1→]n
wherein GlcNpAc is 2-acetamido 2-deoxyglucopyranose, IdoAp is idopyranosyluronic acid and S is sulfate, and n is 1 to 1000 that is effective in inhibiting an increase in the volume or mass of a tumor in a host in need of such inhibiting effect.

17. The composition of claim 16 wherein n is 4 to 500.

18. The composition of claim 16 wherein n is 51 to 100.

19. The composition of claim 16 wherein n is 4 to 100.

20. The composition of claim 16 wherein n is 4 to 50.

21-26. (canceled).
